(19) United States
(12) Patent Application Publication
(10) Pub. No.: US 2020/0337627 A1
(43) Pub. Date: Oct. 29, 2020

(54) COGNITIVE HEALTH ASSESSMENT SYSTEM

(71) Applicants: Massachusetts Institute of Technology, Cambridge, MA (US); Lahey Clinic Foundation, Inc., Burlington, MA (US)

(72) Inventors: Dana L. Penney, Weston, MA (US); Randall Davis, Weston, MA (US)

(21) Appl. No.: 16/859,078

(22) Filed: Apr. 27, 2020

Related U.S. Application Data
(60) Provisional application No. 62/838,887, filed on Apr. 25, 2019.

(51) Int. Cl. A61B 5/00; G09B 19/00; A61B 3/113

(52) U.S. Cl. CPC A61B 5/4088; G09B 19/00; G06F 3/03545; A61B 5/7264; A61B 5/742; A61B 3/113

(57) ABSTRACT

A method of determining a cognitive assessment for a subject includes receiving input position data associated with input provided by the subject during a time that the subject is responding to a cognitive test, receiving gaze position data associated with a gaze of the subject during the time that the subject is responding to the cognitive test, and determining a cognitive assessment for the subject based at least in part on the input position data and the gaze position data.

### CROSS-REFERENCES TO RELATED APPLICATIONS

This application claims the benefit of U.S. Provisional Application No. 62/838,887 filed Apr. 25, 2019, the contents of which are incorporated herein by reference.

### BACKGROUND OF THE INVENTION

In the field of cognitive assessment, specialized tests are used to assess the cognitive health of subjects. For example, a subject is instructed to undertake a task that is carefully designed to exercise certain cognitive functions. The subject's performance on the task provides insights that a professional (e.g., a medical professional) can use to assess the subject's cognitive health. Examples of cognitive capabilities that are commonly assessed are memory, learning, inductive reasoning, and decision making.

Examples of cognitive tests include the clock drawing test, the Montreal Cognitive Assessment (MoCA), the Mini-Mental State Exam (MMSE), and the Mini-Cog.

### SUMMARY OF THE INVENTION

Generally, cognitive assessment tests instruct a subject to answer a question or to perform a task. The subject then responds by answering the question or performing the task. The subject's response is analyzed to assess the subject's cognitive capabilities.

For at least some cognitive assessment tests, additional information related to a subject's cognitive capabilities can be obtained by monitoring actions of the subject while they are formulating their response to a cognitive assessment test.

Aspects described herein concurrently monitor both a gaze of a subject and an input position (e.g. a position of a stylus or finger on a screen or other suitable input device) as the subject formulates their response. As is described in greater detail below, doing so provides additional information related to the subject's cognitive capabilities that can be used when assessing the subject's cognitive health.

In a general aspect, a method of determining a cognitive assessment for a subject includes receiving input position data associated with input provided by the subject during a time that the subject is responding to a cognitive test, receiving gaze position data associated with a gaze of the subject during the time that the subject is responding to the cognitive test, and determining a cognitive assessment for the subject based at least in part on the input position data and the gaze position data.

Aspects may include one or more of the following features.

The input position data and the gaze position data may be aligned to a common timeline. The input position data may include a time series of input positions and the gaze position data may include a time series of gaze positions. Determining the cognitive assessment may include processing the input position data and the gaze position data using a parameterized transformation. The method may include pre-processing the input position data and the gaze position data according to data characterizing the cognitive test prior to using the parameterized transformation. The parameterized transformation may include a neural network.

The cognitive test may be a symbol-digit test. Determining the cognitive feature may include measuring a period of time for which the subject gazed at a stimulus symbol in the symbol-digit test, detecting whether the subject gazed at a key of the symbol-digit test, detecting whether the subject gazed at a prior stimulus item, detecting whether the subject gazed at a position or a feature of the displayed test, measuring a period of time for which the subject gazed at a key of the symbol-digit test, measuring a period of time for which the subject obtained a correct pairing or an incorrect pairing, or any combination thereof. The symbol-digit test may include a symbol-digit decoding task. The symbol-digit test may include a digit-digit copying task.

The cognitive test may be a maze test. Determining the cognitive feature may include measuring a position of the subject's gaze, comparing the position of the subject's gaze to a position of an input provided by the subject, determining whether the subject pauses, determining whether the subject retraces a path, or any combination thereof. The maze test may be a no-choice test. The maze test may include a no-choice subtest. The maze test may be a choice test. The maze test may include a choice subtest.

The cognitive test may be displayed on a surface and the writing instrument may be a stylus to which the surface is responsive. The surface may be a tablet computer interface, a wall, or a virtual surface. The cognitive test may be displayed on a physical or electronic page and the writing instrument may be a digitizing pen. The cognitive test may include subtests of varying cognitive loads. The method may include changing a visual appearance of a stimulus of the cognitive test. Changing the visual appearance of the stimulus may include producing a change in cognitive load or perceived cognitive load. The method may include determining an impact of the changed cognitive load based on a detected gaze.

The method may include displaying the cognitive test to a subject. Determining the cognitive assessment for the subject based at least in part on the input position data and the gaze position data may include determining at least part of the cognitive assessment while the subject is still responding to the cognitive test.

In another general aspect, a system for determining a cognitive assessment for a subject includes an input for receiving input position data associated with input provided by the subject during a time that the subject is responding to a cognitive test, an input for receiving gaze position data associated with a gaze of the subject during the time that the subject is responding to the cognitive test, and one or more processors for determining a cognitive assessment for the subject based at least in part on the input position data and the gaze position data.

In another general aspect, a non-transitory computer-readable medium has encoded thereon a sequence of instructions which, when loaded and executed by a processor, causes the processor to perform a method for determining a cognitive assessment for a subject by receiving input position data associated with input provided by the subject during a time that the subject is responding to a cognitive test, receiving gaze position data associated with a gaze of the subject during the time that the subject is responding to the cognitive test, and determining a cognitive assessment for the subject based at least in part on the input position data and the gaze position data.

In another general aspect, a method for determining parameters for a parameterized transformation to be used in a cognitive health assessment system includes receiving input position data associated with input provided by a number of subjects during times that the subjects are responding to a cognitive test, receiving gaze position data associated with a gaze of the number of subjects during the times that the subjects are responding to the cognitive test, receiving cognitive health assessment label data associated with cognitive health assessments determined from a performance of the number of subjects on the cognitive test, and estimating parameters for the parameterized transformation based at least in part on the input position data, the gaze position data, and the cognitive health assessment label data, wherein the parameterized transformation is configured to accept input position data for a subject responding to the cognitive test, gaze position data for the subject responding to the cognitive test, and produce a cognitive health assessment for the subject.

In another general aspect, a method of detecting and measuring a learning process includes displaying a cognitive test to a subject, and, with a device configured to track temporal position of a writing instrument of the subject, such as a stylus or a finger interfacing with a touch screen, obtaining position and time data of responses entered in the cognitive test by the subject. The method further includes, with a device configured to track an eye position of the subject, obtaining position and time data of a gaze of the subject on the displayed cognitive test. A cognitive feature of the subject is determined based on the obtained position and time data of the writing instrument and the eye gaze of the subject.

The cognitive test can be a symbol-digit test. Determining the cognitive feature can include measuring a period of time for which the subject gazed at a stimulus symbol in the symbol-digit test, detecting whether the subject gazed at a key of the symbol-digit test, detecting whether the subject gazed at a prior stimulus item in the symbol-digit test, detecting whether the subject gazed at a position or a feature within the test, measuring a period of time for which the subject gazed at a key of the symbol-digit test, measuring a period of time for which the subject obtained a correct pairing or an incorrect pairing, or any combination thereof. The symbol-digit test can include a symbol-digit decoding task, a digit-digit decoding task, or a combination thereof.

Alternatively, the cognitive test can be a maze test. Determining the cognitive feature can include measuring a location of the subject's gaze, comparing the location of the subject's gaze to a position of the writing instrument, determining whether the subject pauses, determining whether the subject retraces a path, or a combination thereof. The maze test can be a no-choice test or can include a no-choice subtest. In addition, or alternatively, the maze test can be a choice test or can include a choice subtest.

The cognitive test can be displayed on a surface, such as a touch screen surface of a tablet computer or a virtual surface, and the writing instrument can be a stylus to which the surface is responsive. The cognitive test can be displayed on a physical or electronic page, or in virtual or augmented reality. The writing instrument can be a digitizing pen.

The cognitive test can include subtests of varying cognitive loads. A visual appearance of a stimulus of the cognitive test can be changed. For example, the stimulus can be changed in a manner that produces a change (e.g., an increase or decrease) in cognitive load or perceived cognitive load. An impact of a changed cognitive load can be detected by eye tracking.

Aspects may have one or more of the following advantages.

Aspects described herein advantageously improve upon conventional cognitive health assessment techniques by tracking the subject's input and gaze over time to obtain insights into cognitive processes employed by the subject when completing cognitive health assessment tests.

Other features and advantages of the invention are apparent from the following description, and from the claims.

### BRIEF DESCRIPTION OF THE DRAWINGS

FIG. 1 is a cognitive health assessment system.

FIG. 2 is a completed translation task of a symbol-digit test.

FIG. 3 is a completed copying task of a symbol-digit test.

FIG. 4 is a completed delayed recall task of a symbol-digit test.

FIG. 5 is a sample gaze pattern for a subject completing the translation task of FIG. 3.

FIG. 6 is a gaze pattern indicating learning for a subject completing the translation task of FIG. 3.

FIG. 7 is a gaze pattern indicating use of short-term memory for a subject completing the translation task of FIG. 3.

FIG. 8 is a completed calibration maze.

FIG. 9 is a completed no-choice maze.

FIG. 10 is a completed choice maze.

FIG. 11 is a gaze pattern showing a subject working ahead when completing the choice maze of FIG. 10.

FIG. 12 is a gaze pattern leading a stylus position by a normal amount for a subject completing the choice maze of FIG. 10.

FIG. 13 is a gaze pattern leading a stylus position by an abnormally small amount for a subject completing the choice maze of FIG. 10.

FIG. 14 is a gaze pattern after a subject completing the choice maze of FIG. 10 makes a mistake.

FIG. 15 is a training system.

### DETAILED DESCRIPTION

### 1. Overview

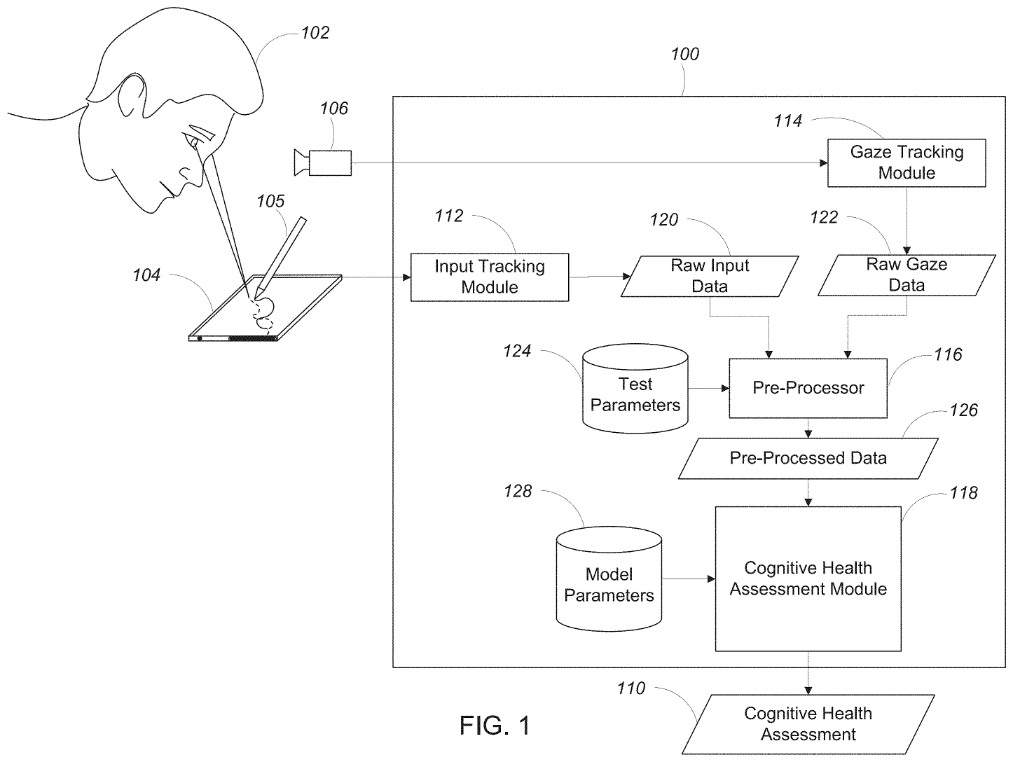

Referring to FIG. 1, a cognitive health assessment system 100 administers a cognitive health assessment test to a subject 102 using a computing device such as a tablet 104. As the subject 102 works on completing the test, the tablet 104 records a position of a stylus 105 (or finger) on the tablet's touch screen over time and one or more cameras 106 record the subject's gaze over time.

The cognitive health assessment system 100 processes the position of the stylus over time and the recording of the subject's gaze (e.g., a location on the tablet being viewed by the subject) over time to determine a cognitive health assessment 110 for the subject 102. As is described in greater detail below, by accounting for the subject's gaze and the position of the stylus over time, the cognitive health assessment 110 is based not only on the subject's response to the task, but also on additional information related to the process used by the subject 102 to arrive at the response.

### 2. Cognitive Health Assessment System

The cognitive health assessment system 100 includes an input tracking module 112, a gaze tracking module 114, a pre-processor 116, and a cognitive health assessment module 118.

In operation, the position of the stylus over time is provided to the input tracking module 112, which processes the position to generate raw input data 120 including a time series of positions of the stylus on the screen of the tablet 104. The recording of the subject's gaze over time is provided to the gaze tracking module 114, which processes the recording (e.g., a video recording) to generate raw gaze data 122 including a time series of gaze positions (e.g., (x, y) locations) on the screen of the tablet 104. In general, the raw input data 120 and the raw gaze data 122 are synchronized to a common timeline, such that for any given position of the stylus on the tablet screen, the position of the subject's gaze on the tablet screen is known.

The raw input data 120 and the raw gaze data 122 are provided to the pre-processor 116 along with test parameters 124. The pre-processor 116 processes the raw input data 102 and the raw gaze data 122 using the test parameters 124 to generate pre-processed data 126. Very generally, the test parameters 124 characterize features and/or a structure of a specific test being administered to the subject 102. As is described in greater detail below, the tests administered to the subject 102 by the cognitive health assessment system 100 include, but are not limited to, symbol-digit tests and maze following tests. In those cases, the test parameters 124 include information such as (x,y) locations of symbols/digits, maze decision points on the screen of the tablet 104, positions of the walls of the maze, or locations and contents of a number of cells spanning the screen of the tablet 104.

In some examples, the pre-processor 116 generates the pre-processed data 126 by processing the raw input data 120 and the raw gaze data 122 according to the test parameters 124 to extract one or more fixed-length feature vectors (e.g., descriptors comprising vectors or arrays of numbers) from the raw data (including both the raw input data 120 and the raw gaze data 122). In some examples, a series of fixed-length feature vectors is extracted by first segmenting the raw data according to the raw input data 120 (e.g., according to a cell position of the stylus on the screen of the tablet 104). A fixed-length feature vector for each segment is then determined. For example, a fixed-length feature vector could include an identifier (e.g., an index) of the symbol pointed to by the stylus and a histogram representing an amount of time the subject's gaze fell on each cell on the screen of the tablet 104. To the extent that there are a fixed number of segments in the test, the series of fixed-length feature vectors together form a fixed-length input to further processing described below.

In other examples, for example when there is a variable length series of segments, a sequence-to-fixed length transformation may be used, and such a transformation may be predefined, or may be learned based on training data. For example, as described below, a recurrent neural network (e.g., a Long Short Term Memory, LSTM, network) may be used to transform the sequence of segment features to form a combined fixed length representation.

The cognitive health assessment module 118 receives the pre-processed data 126 (i.e., a fixed-length output of the pre-processing of the input stylus and gaze data) and a set of model parameters 128. The cognitive health assessment module 118 processes the pre-processed data 126 using the set of model parameters 128 to generate the cognitive health assessment 110. The assessment may represent a prediction of one of a predefined set of classes, or a (posterior) distribution over the classes given the input data, or may represent a score or degree for a characteristic of the subject, for example, a score indicating the degree of a particular type of impairment or condition or the likelihood that the subject has the particular impairment or condition. In some examples, the cognitive health assessment module 118 is a classifier that is parameterized according to the set of model parameters 128, which are determined in a previous training step (described in greater detail below). In some examples, the cognitive health assessment module 118 is implemented as a neural network (e.g., a “deep” neural network). In other examples, cognitive health assessment module 118 is implemented as another type of classifier (e.g., a support vector machine, nearest neighbor classifier, etc.) or parameterized predictive model. In some alternatives, the transformation of the variable length sequence of inputs and the health assessment stage may be combined into a single component, for example, being a jointly trained recurrent neural network.

As is described in greater detail below, the resulting cognitive health assessment 110 includes information related to the subject's cognitive health including but not limited to the subject's learning abilities and processes, decision making processes, logical reasoning processes, and short and long-term memory abilities.

### 3. Cognitive Health Assessment Tests

As is mentioned above, the cognitive health assessment system 100 administers cognitive health assessment tests to subjects, where the cognitive health assessment tests include symbol-digit tests and maze following tests. These tests are designed with specific stimuli, administration, and behavior capture features that enable the system 100 to distinguish specific cognitive and motor functions (graphomotor, eye movement, etc.) under particular performance conditions (speed, incidental learning, implied instructions, etc.). This enables comparisons that enable the subject to be used as their own control, in addition to population normative standards. This also enables the test to provide consistent measurements under transient state changes like fatigue, depression, test taking attitude, and sandbagging. This is accomplished by having the subject do specific aspects of the same task that are combined to create conditions of different cognitive loads with the same physical load.

Performance under light cognitive load and a given physical load provides a baseline measurement while performance under heavier cognitive load and the same given physical load generally measures maximal performance. Changes in performance across features and conditions under different levels of load informs diagnosis and treatment. The comparison of, for example, movement speed under lighter and heavier cognitive load allows the system 100 to separate out factors that may be due to physical condition versus those due to cognitive conditions.

For both the symbol-digit test and the maze following test, the system 100 requires the subject to complete the same physical task twice, under different feature and task conditions that impact cognitive load. As a result, the tests elicit physical responses from the subjects that can be used to infer characteristics of the subjects' cognitive health.

### 4. Symbol Digit Test

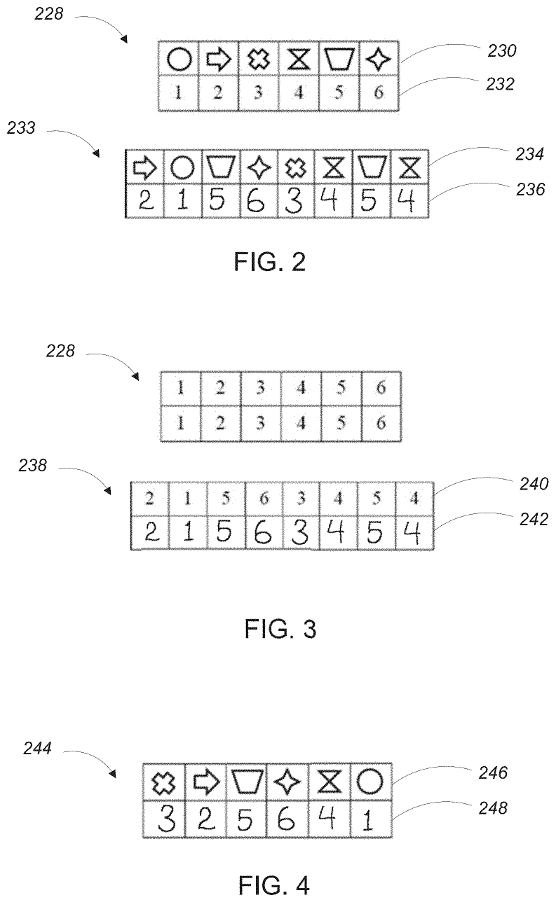

As is mentioned above, one example of a cognitive health assessment test is the “symbol-digit test.” Referring to FIGS. 2-4, one simplified example of the symbol-digit test has three sections: a translation task section, a copying task section, and a delayed recall task section which are presented to a subject sequentially. In some examples, both the translation task section and the copying task section also including “warmup” exercises that allow the subject to practice the tasks.

Referring to FIG. 2, first the translation task is presented to the subject. In the translation task, a key 228 including a number of symbols 230, each associated with corresponding digit 232, is presented to the subject. At the same time, a translation task section 233 is presented to the subject. The translation task section 233 includes a number of symbols 234 selected from the key 228, each associated with a corresponding empty box 236. The translation task requires that the subject reference the key 228 to fill in the empty boxes 236 with the digits corresponding to the symbols 234. The translation task shown in FIG. 2 shows the task section completed.

Referring to FIG. 3, the copying task is then presented to the subject. For the copying task, the key 228 is again presented to the subject (to keep the page layout, spatial, and motor aspects consistent with the translation task). At the same time, a copying task section 238 is presented to the subject. The copying task section 238 includes a number of digits 240, each associated with a corresponding empty box 242. The copying task requires that the subject copy the digits 240 shown in the copying task section 238 into their corresponding empty boxes 242. The copying task shown in FIG. 3 shows the task section completed.

Referring to FIG. 4, the delayed recall task is then presented to the subject. For the delayed recall task, a delayed recall task section 244 is presented to the user without the key 228 being presented. The delayed recall task section 244 includes a number of symbols 246 selected from the key 228, each associated with a corresponding empty box 248. The delayed recall task requires that the subject fill in the empty boxes 248 with the digits corresponding to the symbols 246 from memory, without being able to reference the key 228. The delayed recall task shown in FIG. 4 shows the task section completed.

In general, the above-described symbol-digit test is administered twice in succession, where the subject is unaware that they will have to complete the delayed recall section in the first administration of the test. By repeating the test, the subject's experience with the test can be used as a test feature (e.g., in healthy subjects, better performance is expected on the second repetition).

### 4.1 Test Administration Strategies and Inferences

When administering the test, the system 100 instructs the subject to “work as quickly and accurately as possible,” suggesting that the test is measuring cognitive motor processing speed. Unknown to the subject, the test also measures incidental memory via the delayed recall section.

When completing the delayed recall section, successfully filling in any of the boxes correctly is an indicator of learning and hence another sign of cognitive health. Information is also obtained from the order and speed with which the boxes are filled in. That information is provided by the data from the stylus, which, in some examples, time stamps every (x,y) position that it visits on the screen of the tablet 104. This type of information provides insights as to which symbols were easier to recall, as they may get answered first, more quickly, or both. A time delay between pen strokes provides information as to how much time the subject spends thinking but not writing, while they attempt to recall the digits for the next symbol.

After the first administration of the test, the subject is told that the next test is identical to the one they just took, and exactly the same instructions are given. The subject's experience during the first administration of the test, plus the indication that the same test is being given again, lets them know that the delayed recall section will appear again. This test administration approach enables measurement of aspects of learning from experience, and cognitive strategies used by the subject under different expectations. Strategies used to maximize speed are not usually the best strategies for learning. How a subject adapts to the changing constraints enables measurement of not only performance on the test, but also ability to learn from experiences. Learning from experience is yet another sign of cognitive health.

In some examples, performance on the delayed recall conditions is sensitive to subtle cognitive impairment in subjects at risk for neurodegenerative disorders such as Alzheimer's who otherwise perform normally on standard tests. Performance provides predictive indications of future impairment in subjects that appear cognitively healthy.

In some examples, changes in response speed (measured as pen/stylus movement and/or gaze movement) can be used to infer cognitive load. Pupil size, an indicator of the perceived difficulty of a task, can also be measured, where the more difficult a task seems, the larger the subject's pupils become. Measurements such as changes in response speed and pupil size may be used to determine the subject's perceived level of difficulty. That perception can be compared under different testing conditions (i.e., comparing the subject to themselves). That perception can also be compared to relative level of perceived difficulty to norms established from testing healthy controls.

In general, any successful performance by a subject on the first administration of the delayed recall task is referred to as incidental learning, because healthy subjects learn some of the associations while doing the translation task, even though they don't know they will be tested to see whether they have memorized them.

For the second test administration, performance on the delayed recall task is informed by prior experience with the test. This changes the delayed recall into an implied learning task, because the subjects should infer the recall condition is coming even if not explicitly stated in the instructions. The lack of behavior change by the subject on the second administration of the test indicates a failure of the subject to adjust to the task change and is an indicator of cognitive impairment.

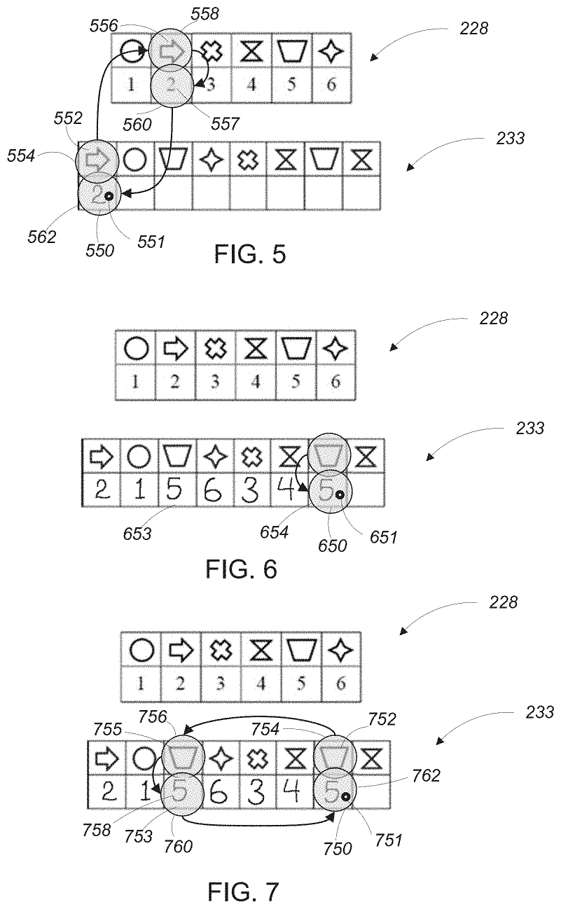

In some examples, eye tracking shows that during the early part of the translation task, subjects scan the key in order to look up the associated digit. Such a scenario is described in greater detail below with reference to FIG. 5. Later, when the subject writes down an answer without looking at the key, there is evidence that they have, for the moment at least, learned that association. The system 100 detects this “learning in real time” as the moment that the subject establishes an association between a symbol and its corresponding digit. Learning is an important sign of cognitive health. Such a scenario is described in greater detail below with reference to FIG. 6.

Also illustrated below, a subject may gaze at boxes further back in the test that they have already filled in in order to find the associated digit. Doing so saves some effort as compared to looking at the key. This successful use of short-term memory in recalling recent appearances of a symbol is a sign of cognitive health. Such a scenario is described in greater detail below with reference to FIG. 7. Further, eye-tracking enables the system 100 to assess the efficiency of this strategy: does the subject find the previous instance right away, or have trouble locating it? Does looking back end up taking more time than referring to the key? Inefficient look-back is a sign of cognitive impairment.

The ability to decide to refer back to one of one's own responses, rather than check the key, is yet another sign of cognitive health. For the subject to make a change in their approach to the test, the subject also has to multi-task, i.e., strategize and make a decision while taking the test. This too is a sign of cognitive health.

### 4.2 Examples

Throughout both administrations of the symbol-digit test, raw input data and raw gaze data are collected. The collected data is pre-processed in the pre-processor and then processed in the cognitive health assessment module 118 to generate the cognitive health assessment 110. The following examples illustrate just a few of the many types of inferences that can be made from the raw input and gaze data.

### 4.2.1 Exemplary Gaze Pattern

Referring to FIG. 5, when completing the translation task for the symbol-digit test, the subject begins by placing their stylus 551 in a first empty box 550 of the translation task section 233 in anticipation of writing a digit into the empty box 550. At that time, the subject's gaze is directed at a symbol 552 (e.g., the right arrow symbol) above the empty box 550. A first gaze location 554 is recorded as the subject gazes at the symbol 552.

The subject's gaze then moves to the key 228 and finds the symbol 556 in the key 228. A second gaze location 558 is recorded as the subject gazes at the symbol 556 in the key 228. The subject's gaze then moves to the digit 557 (i.e., “2”) associated with the symbol 556 in the key 228. A third gaze location 560 is recorded as the subject gazes at the digit 557. The subject's gaze then moves back to the empty box 550, where the subject writes the digit (i.e., “2”) into the empty box 550. A fourth gaze location 562 is recorded as the subject gazes at the empty box and writes the digit.

The stylus and gaze locations for the above-described sequence of actions represent one example of a segment of raw data that can be transformed to a fixed-length feature vector by the preprocessor 116 and then processed in the cognitive health assessment module 118 as part of determining the cognitive health assessment 110. In the example above, the stylus and gaze locations indicate a normal cognitive process for completing the translation task.

### 4.2.2 Gaze Pattern Indicating Learning

Referring to FIG. 6, in another example of a subject completing the translation task for the symbol-digit test, the subject places their stylus at a location 651 in a seventh empty box 650 of the translation task section 233 in anticipation of writing a digit into the empty box 650. At that time, the subject's gaze is directed at a symbol 652 (i.e., a trapezoid symbol) above the empty box 650. A first gaze location 654 is recorded as the subject gazes at the symbol 652.

In this example, the subject has already encountered the trapezoid symbol when filling in a third empty box 653 and was able to memorize that the trapezoid symbol is associated with the “5” digit. Rather than looking to the key to obtain the digit, the subject simply recalls the digit from memory and directs their gaze to the empty box, where they write the digit (i.e., “5”). A second gaze location 654 is recorded as the subject gazes at the empty box and writes the digit.

The stylus and gaze locations for the above-described sequence of actions represent another example of a segment of raw data that can be transformed to a fixed-length feature vector by the preprocessor 116 and then processed in the cognitive health assessment module 118 as part of determining the cognitive health assessment 110. In the example above, the stylus and gaze locations indicate that the subject has learned, in real-time, the association between the trapezoid shape and the digit, “5.”

### 4.2.3 Gaze Pattern Indicating Short-Term Memory Usage

Referring to FIG. 7, in another example of a subject completing the translation task for the symbol-digit test, the subject places their stylus 751 in a seventh empty box 750 of the translation task section 233 in anticipation of writing a digit into the empty box 750. At that time, the subject's gaze is directed at a symbol 752 (i.e., a trapezoid symbol) above the empty box 750. A first gaze location 754 is recorded as the subject gazes at the symbol 752.

In this example, the subject has already encountered the trapezoid symbol when filling in a third empty box 753 and recalls that previous encounter. Rather than looking to the key 228 to obtain the associated with the trapezoid symbol, the subject directs their gaze back to the pervious occurrence of the trapezoid symbol 755. A second gaze location 756 is recorded as the subject gazes at the previous occurrence of the trapezoid symbol 755.

The subject's gaze then moves to the digit 758 (i.e., “5”) that they previously wrote down in the box below the first occurrence of the trapezoid symbol 755. A third gaze location 760 is recorded as the subject gazes at the digit 758. The subject's gaze then moves back to the seventh empty box 750, where the subject writes the digit (i.e., “5”) into the empty box 750. A fourth gaze location 762 is recorded as the subject gazes at the empty box and writes the digit.

The stylus and gaze locations for the above-described sequence of actions represent another example of a segment of raw data that can be transformed to a fixed-length feature vector by the preprocessor 116 and then processed in the cognitive health assessment module 118 as part of determining the cognitive health assessment 110. In the example above, the stylus and gaze locations indicate that the subject has successfully used their short-term memory to retrieve the digit associated with the trapezoid shape without going back to the key 228.

### 5. Maze Following Test

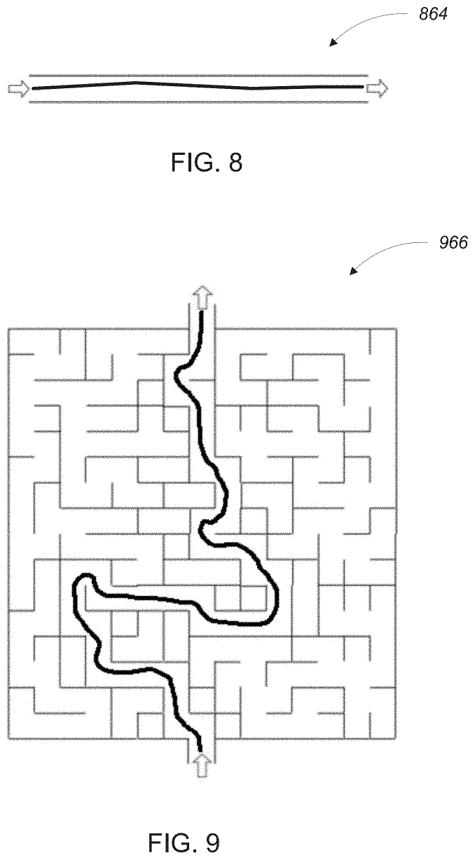

Another example of a cognitive health assessment test administered by the system 100 is the “maze following test.” Referring to FIGS. 8-10, one example of the maze following test has three sections: a calibration maze, a no-choice maze, and a choice maze.

Referring to FIG. 8, the calibration maze 864 is a simple straight path for which the subject told to draw a straight line from one end of the path to the other. The section ensures that the subject understands the task, has the graphomotor ability to perform it, and provides a baseline calibration of their motion speed. Baseline speed may be affected by changes that may occur during the test—such as faster with familiarity or slower with boredom. The calibration maze is performed for each test to provide subject state measures that may affect performance on the other maze sections.

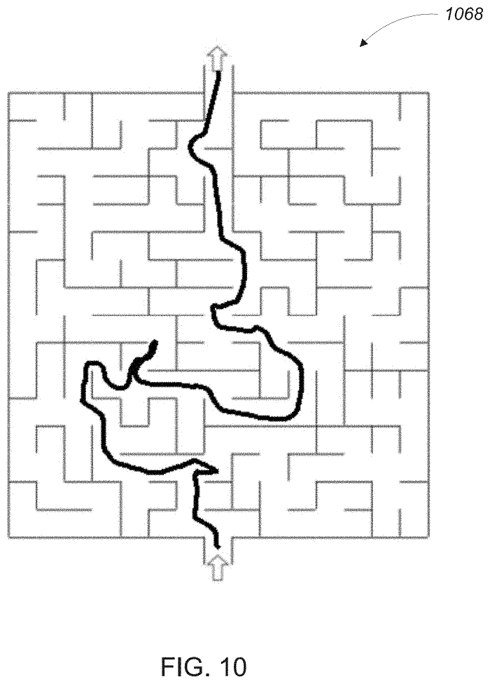

Referring to FIG. 9, the no-choice maze 966 does not include any path choices (unbeknownst to the subject)—there is only a single path to follow. Referring to FIG. 10, the choice maze 1068 includes choices that the subject must make at decision-making junctions.

### 5.1 Test Administration Strategies and Inferences

In general, unbeknownst to the subject, with the exception of the calibration maze, the solutions to all sections of the maze following test are identical.

Several aspects of the maze following test are informative about a subject's cognitive condition. For example, a speed of the stylus on the calibration maze may be used to distinguish subjects with amnestic mild cognitive impairment (aMCI) from healthy controls. Stylus speed alone is also indicative in other sections of the maze following test. For example, slowing down at or around decision points is strongly suggestive of taking time to examine the alternatives. This provides a measure cognitive load, i.e., a way to determine how much difficulty a subject is having at various points in the test.

Gaze tracking provides additional information about the subject's behavior. For example, given only stylus speed and location, inferences can be made about what the subject is doing at that instant, but with gaze tracking, inferences can be made about what the subject is thinking.

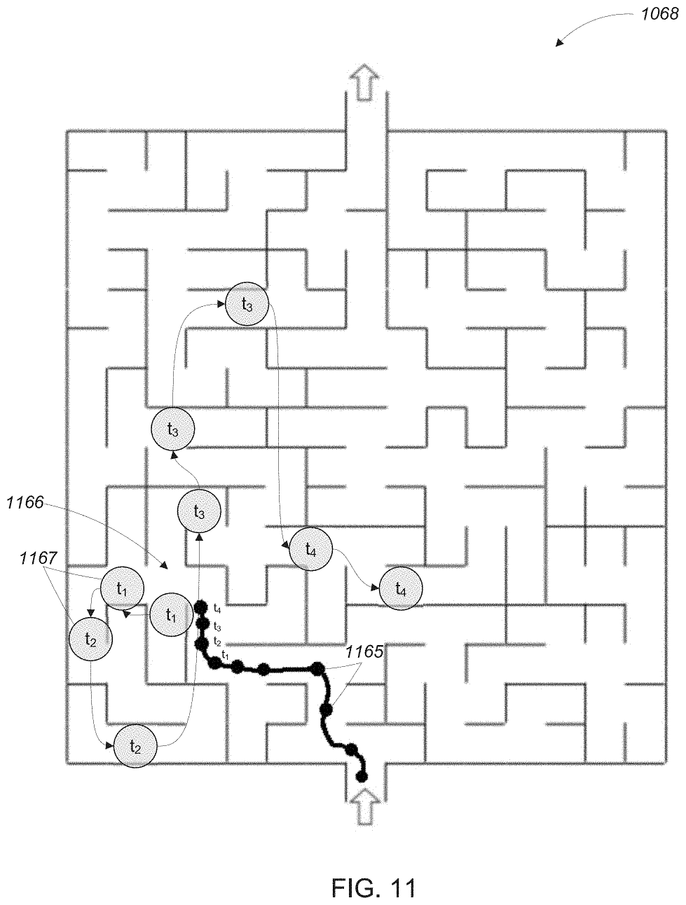

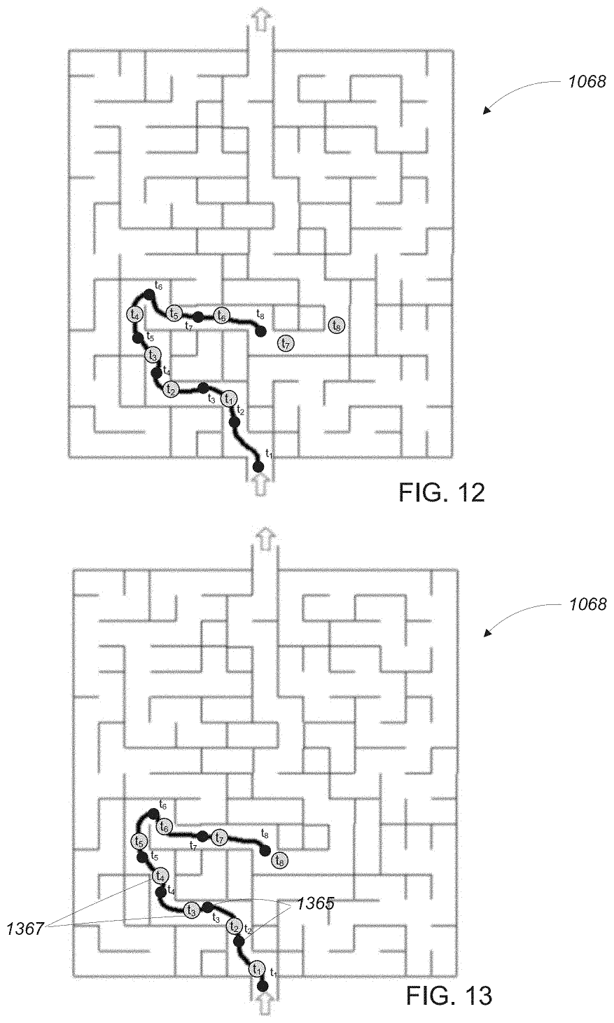

For example, a measured reduction in pen speed before a decision-making junction and a detection of gaze around the upcoming junction can be used to infer that the subject was solving the maze in advance of the pen position. This produces a transient slowdown in stylus speed associated with the decision-making process occurring while the subject was looking at the junction. This is a sign of cognitive health. Such a scenario is described in more detail below with reference to FIG. 11.

More generally the distance between the location of the stylus and the eye gaze position is informative. Having the gaze position ahead of the stylus position suggests normal cognitive capacity—the subject is looking ahead to detect and solve decisions that will have to be made. Such a scenario is described in more detail below with reference to FIG. 12. A reduction in this ability to work ahead is an indication of reduced cognitive ability. Such a scenario is described in more detail below with reference to FIG. 13.

Capturing the moment to moment comparison of stylus position and gaze enables many fine-grained indicators of the level of difficulty experienced by the subject. Knowing how difficult a particular choice is for a subject gives us fine-grained information about their cognitive health.

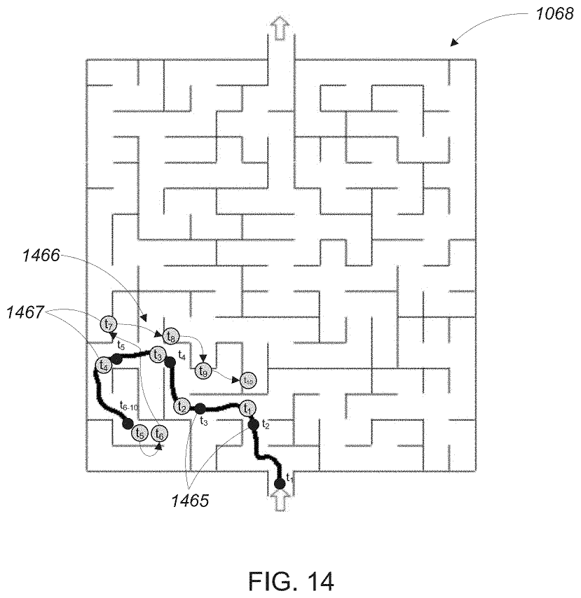

In some examples, the gaze data indicates that the subject suddenly starts looking around extensively. That information combined with stylus position is indicative: if the subject has made a mistake it's normal for them to start trying to figure out where they went wrong. This is another sign of cognitive health. Such a scenario is described in more detail below with reference to FIG. 14.

More detailed analysis of the visual search may also reveal such things as: Is there a methodical search, a failure to look forward, or a bias to one direction (some impaired subjects look mostly to the maze exit and have difficulty making the correct choice when the path leads away from the exit), etc.

In some examples, subjects (frequently those with early Alzheimer's) who are on the correct path, nevertheless have stopped stylus movement and started looking around. Their eye movements indicate that they believe they have made a mistake, when in fact they have not. As one extreme example, some subjects become confused on the no-choice part of the maze following test, even though there are no choices to be made.

In some examples features of maze tests are varied to measure aspects of decision making and cognitive load, including the number of decision making junctions, the complexity of the junctions (2-way, 3-way choices, embedded tiers of choices, etc.) and path lengths. Some features enable comparisons along paths. For example, path lengths can be balanced around decision making junctions—all paths leading into and out of the choice point are all the same length (even incorrect paths). This ensures that all choices including the wrong ones have equal opportunity to be considered—avoiding the risk of one solution being easier simply because it was closer in proximity. This also enables inference of cognitive processes through eye movements during the evaluation of potential pathway solutions.

In some examples, the mazes used in the test are designed to have predetermined levels of difficulty based in part on a complexity of the decision-making junctions and the number of junctions. Easier mazes have fewer decision-making junctions of lower complexity.

In some examples, the mazes have two additional sections that have specific feature that presents the subject with mazes with low and minimal visual clutter. Visual clutter is a hidden form of cognitive load—the perception of the number, length and angles of the lines that are present. Take for example, the subject with Alzheimer's referred to above, who stopped mid-path and backtracked during a no-choice maze. The pen behavior indicates some decision making, pen movement and eye tracking indicates determination of a presumed mistake, and then a corrective action (back tracking). Given there were no obvious decisions to be made, why does the confusion arise? The cognitive load produced by visual clutter may be an important component of the answer (consistent with driving directional confusion in early Alzheimer's). Low and minimal visual clutter test segments measure decision making under conditions of low and now visual clutter, allowing for testing of this hypothesis.

In some examples, measures of decision-making junctions and visual clutter are combined to create choice-point “neighborhoods,” balancing the complexity of the paths adjacent to the correct solution path. This enables capture and measurement of sequences of behavior (eyes, motor, timing) that provide insight into dynamic thinking as it occurs in real time.

### 5.2 Examples

Throughout both administrations of the maze following test, raw input data and raw gaze data are collected. The collected data is pre-processed in the pre-processor and then processed in the cognitive health assessment module 118 to generate the cognitive health assessment 110. The following examples illustrate just a few of the many types of inferences that can be made from the raw input and gaze data.

### 5.2.1 Working Ahead at Decision Point

Referring to FIG. 11, when completing the choice maze section 1068 for the maze following test, the subject begins by moving the stylus through the maze quickly as is evidenced by relatively large spaces between recorded locations 1165. But as a subject approaches a decision point 1166, they begin to move the stylus more slowly as they consider the decision, as is evidenced by relatively smaller spaces between the recorded locations 1165.

Furthermore, when the stylus is at the recorded stylus locations associated with times t₁-t₄ near the decision point 1166, the subject's gaze locations 1167 associated with times t₁-t₄ are distributed around the decision point indicating that the subject is looking ahead to determine which path from the decision point is the best choice. This type of working ahead indicates a healthy cognitive behavior.

### 5.2.2 Stylus Leading Gaze—Healthy

Referring to FIG. 12, when completing the choice maze section 1068 for the maze following test, the subject moves the stylus through the maze while directing their gaze ahead of the stylus position in the maze. In this example, the subject's gaze locations 1267 lead the stylus locations 1265 by about 1.5 recorded locations in FIG. 12 (i.e., the subject's gaze is directed past the stylus location associated with time t₃ (in the future) while the stylus is located at the stylus location associated with time t₂). The scenario in FIG. 12 illustrates a healthy subject working ahead.

### 5.2.3 Stylus Leading Gaze—Impaired

Referring to FIG. 13, when completing the choice maze section 1068 for the maze following test, another subject moves the stylus through the maze while directing their gaze ahead of the stylus position in the maze. In this example, the subject's gaze locations 1367 lead the stylus locations 1365 by very little (i.e., the subject's gaze is directed just in front of stylus location associated with time t₂ while the stylus is located at the stylus location associated with time t₂). The scenario in FIG. 13 illustrates a possibly cognitively impaired subject attempting to work ahead.

### 5.2.4 Scanning after Making a Mistake

Referring to FIG. 14, when completing the choice maze section 1068 for the maze following test, a subject moves the stylus through the maze and makes a mistake at a decision point 1466, leading them down a dead-end path. When the stylus reaches the stylus location 1465 associated with time t₆, the subject realizes their mistake. While the stylus remains substantially in one location for a number of time points (i.e., times t₆-t₁₀) the subject's gaze locations 1467 at those time points moves back through the maze to determine where they went wrong. The scenario in FIG. 14 illustrates a cognitively healthy subject's reaction to making a mistake in the maze following test.

### 6. Cognitive Health Assessment Module Training

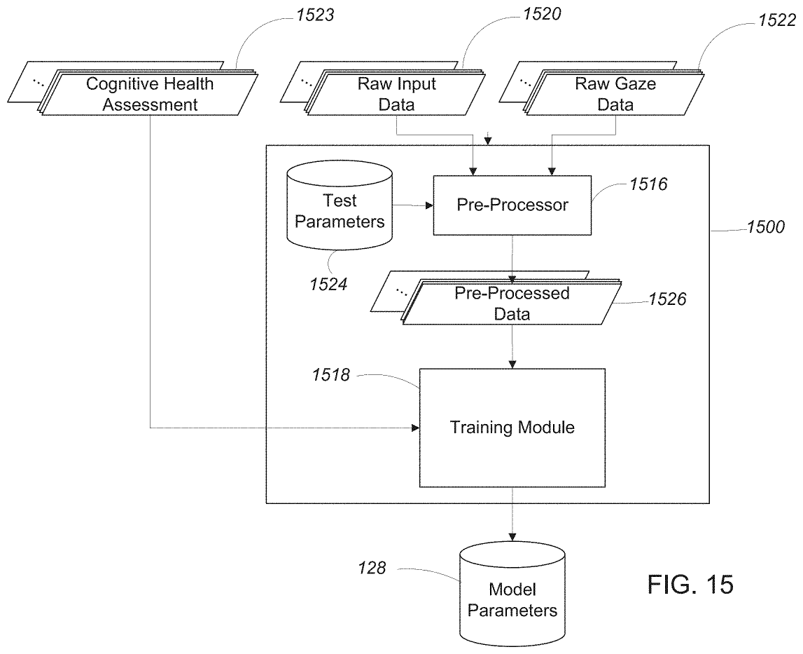

Referring to FIG. 15, as is mentioned above, in some examples the cognitive health assessment module 118 is a transformation such as a neural network that is parameterized by model parameters 128. A training system 1500 is configured to receive input data including triplets of raw input data 1520, raw gaze data 1522, and cognitive health assessment labels 1523 associated with the raw input and gaze data and to process the input data to determine the model parameters 128.

The training system 1500 includes a pre-processor 1516 and a training module 1518. The raw input data 1520 and the raw gaze data 1522 are provided to the pre-processor 1516 along with test parameters 1524. The pre-processor 1516 processes the raw input data 1520 and the raw gaze data 1522 using the test parameters 1524 to generate pre-processed data 1526. As was the case with the test parameters 124 in FIG. 1, the test parameters 1524 characterize features and/or a structure of a specific test being administered to the subject 102.

The pre-processor 1516 generates the pre-processed data 1526 by processing the raw input data 1520 and the raw gaze data 1522 according to the test parameters 124 to extract one or more fixed-length feature vectors from the raw data (including both the raw input data 120 and the raw gaze data 122), as is described above with reference to the pre-processor 116 of FIG. 1.

The pre-processed data 1526 is provided as input to the training module 1518 along with the cognitive health assessment labels 1523. The training module 1518 processes its inputs to generate the model parameters 128. In some examples, the training module 1518 processes its inputs using an optimization algorithm such as a gradient descent algorithm (or any other suitable optimization algorithm known in the art) to determine the model parameters 128.

### 7. Embodiments and Alternatives

A description of example embodiments and alternatives follows.

### 7.1 Symbol-Digit Test

Consider a learning task of the sort traditionally used in psychological testing. The subject might be given a form of the sort shown in FIG. 2, where the task is fill-in-the-blanks with the digit corresponding to the symbol as shown in the key at the top.

The subject may next be given another task, and then given a blank version of the key with the symbols shuffled and asked to fill in the appropriate numbers from memory. This is a technique called delayed recall, which measures learning by seeing how well the subject has learned the symbol-digit pairings.

The above-described technique of delayed recall can provide a useful measure of what the subject has learned but does not indicate when or how the subject learned. An embodiment provides, among other things, a means for determining when and how the subject learned.

### 7.1.1 Example Embodiment

An example embodiment of the present invention is a system and method of detecting and measuring learning processes in real-time.

The example embodiment includes four components that interact synergistically:
a) a designed test;
b) an input device for writing that simultaneously tracks a position of a writing instrument with spatial and temporal accuracy, e.g., via a stylus and electronic tablet, a digitizing ballpoint pen, finger on a touch screen, or other such device;
c) a tracking device for tracking the subject's gaze with spatial and temporal resolution, enabling the determination of where on the test form they are gazing; and
d) a processing module for analyzing a position of said input device for writing and tracking device for eye position data, enabling such measurements as:
   a. how long the subject gazed at a stimulus symbol,
   b. whether and how long the subject gazed at the key to determine the pairing,
   c. if the subject gazed at the key, how quickly the subject found the correct figure,
   d. etc.

The terms “gaze” and “gazing” as used herein may alternatively be referred to as “look” or “looking.” A subject may gaze, or look, at a region of the display of the test for a period of time, such as for a fraction of a second, one or more seconds, or one or more minutes.

As used herein, the term “writing instrument” includes any instrument with which a subject may enter a response to a test, including, for example, a stylus, a pen, a digitizing pen, a finger, or other device manually operable by the subject.

### 7.1.2 The Test

The test forms can have properties that facilitate learning and enable the manifestation, quantification and measurement of multiple learning processes. These properties include a novel design that uses designed and paced exposure to stimuli and using stimuli that are easily learned. An example version uses primary shapes and 3 digits (0, 1, 2) in combination.

The use of digits (0, 1, 2) and primary shapes can enable:
a. Detecting and measuring effects of embedded strategies and interference on learning strategies and memory retention—there are 3 pointy shapes, 3 single digit numbers, and 3 double digit numbers formed by recombining the single digits;
b. Life spectrum assessment—the use of primary and secondary shapes and numbers that are learned first in child development, regardless of language/culture, enables assessment of the development of learning strategies and measurement of memory in children; and
c. Education/Cultural/Language neutral assessment—subjects can use their own native language to name the shape-number pairs, enabling global application

The designed and spatially paced exposure to stimuli can include:
a. Exposure to each symbol every six response squares, randomized within each six-square increment, while ensuring that no symbol is presented twice in succession, maintaining an equal exposure;
b. The creation of multiple equivalent forms that recombine the original stimuli, making possible repeated testing without specific item pair bias (i.e., learning pairs from prior exposure).

The use of a limited number of pairs (6) can enable:
a. Sufficient number of repeated exposures to make possible learning even in subjects with memory impairment;
b. Measuring memory change, whether improvement or decline.

The page layout:
a. Enables an entire test to be contained on one page, yet, when folded, the first section covers and thereby hides the delayed recall.
b. Uses the second side similarly, to cover the answer key during recall.

### 7.1.3 Example Testing Procedure

An example test design includes two halves: the first half of the test is the symbol-digit “decoding.” The second half of the test is a digit-digit copying task, where the task is simply to copy the digit in the top half of the cell into the bottom half. Unknown to the subject, the answers to the two halves of the test are identical.

The copying task provides a useful measurement of the subject's movement speed. This differs even among normal individuals and may be substantially different for impaired persons. Given this measurement as a baseline of the subject's movement speed, a system, such as a tablet computer, configured as a test platform can then distinguish e.g., what part of the subject's speed is due to cognitive load (having to look up or remember the symbol-digit pairings) versus due to simple muscle speed. In effect, the test uses each subject as the subject's own control.

The form also has a delayed recall portion: once the subject is done with the digit-digit copying task, the subject is asked to recall, from memory, a pairing of the symbols and digits used on the first half of the test.

The form is designed to capture a wide range of learning strategies, including shape and number selection and pairing. The form design enables creation of specific learning association strategy scales. For example, one version has numbers in ascending order, with double digits paired with “pointy” shapes—enabling a chunking strategy that may facilitate learning. Other chunking combinations are possible with these specific stimuli and test design.

The test design may include giving the subject the identical form twice in a row. This enables an assessment of implied learning. Current assessment tools may assess incidental learning, in which memory testing is a surprise (as in a typical delayed recall test) or explicit learning in which the subject is told the test is for memory, and, hence, attempts to memorize the associations.

The idea of using a second administration of the same test with the specific instructions presented creates an implied memory paradigm. The subject needs to recall his or her prior experience with the first administration, then apply reasoning and predict that there will be a memory recall. A subject who makes this inference has an opportunity to adapt his or her performance to improve learning.

The implied memory paradigm measures processes of learning and memory that have higher ecological validity. The processes are, for instance, more like real world experience than telling someone explicitly to learn something in preparation for a memory test.

This test design also enables measuring learning from exposure, aspects of reasoning, and flexibility of learning strategies.

### 7.1.4 The Data

The combined data from digital pen and eye tracking can enable a variety of important measures of learning. As one example, there is strong evidence that a subject has learned an association if the subject can fill in a blank in FIG. 4 correctly without having to refer to the key at the top of the form. The system on which the subject is taking the test determines whether the subject referred to the key from the eye tracking data.

As the stimuli contain numerous instances of each stimulus figure, and the eye tracking occurs in real time, the system may determine exactly when the subject did not need to refer to the key, providing real-time detection of learning.

It is expected that the learning will be a gradual process, hence, the subject may be able to retrieve the correct answer from memory at one point, and further on in the test, may have to refer back to the key. Detection of the subject's gaze can thereby provide for monitoring the progress of learning, rather than treating learning as a binary state.

It is expected that there will be multiple strategies involved in taking the test that will likewise reveal aspects of the subject's cognitive status. While the key shows the pairing of symbol and digit, the test form can also include pairings in the form of blanks already filled in by the subject. The test can provide for detection of when the subject “looks up” the pairing by referring back to a cell they have previously filled in, rather than looking in the key.

It is also believed that a decline in the ability to make use of incidental learning may be very early evidence of cognitive decline, of the sort that occurs early in diseases such as Alzheimer's. While memory failure is a known early sign of cognitive decline, this test provides the ability to study the learning process, whose decline is likely to be a predecessor to memory loss. This in turn means the test may provide some of the earliest detection of symptoms related to Alzheimer's.

The test metrics can depend on combinations of graphomotor and visual features that are precisely defined operationally, to enable automated assessment of cognitive functions captured by the test. Consider, as one example, an operational definition of the time when the subject is not writing (and hence may be resting the pen on the page or looking up to the answer key). The digital capture of writing behavior enables the detection and capture of micro movements, even when the subject does not appear to the observer to be writing and they intend to hold the pen still. But holding the pen (or anything else) perfectly still is in fact quite difficult, particularly for those with some variety of tremor. Hence the metrics can specify precisely how little movement is required in order to classify the pen as not writing.

### 7.2 Maze Following Test

Consider a maze task of the sort traditionally used in psychological testing. The subject might be given a form of the sort shown in FIG. 10 and asked to find a path from start to finish. Typically, there is a set of mazes of increasing difficulty, and testing continues until the subject fails to find a solution with a threshold amount of time. Typically, the only outcome from the test is what level maze they completed and what paths they drew, i.e., a final result of their efforts.

Subjects have been administered maze tests with use of a stylus that measures a position of the stylus on the page with spatial and temporal accuracy. Because each data point is time-stamped, both the final drawing and the graphomotor behaviors that produced it (e.g., the pauses, backtracking, etc.) can be captured. This has produced a number of interesting capabilities and discoveries about human behavior. (See U.S. Pat. No. 9,895,9085, the entire contents of which are incorporated herein by reference).

One further insight is the counter-intuitive observation that, for some subjects, decision points in the maze may not be the sole source of cognitive difficulty. Paths alone, even without choices, can present cognitive load to certain subjects.

While knowing what a subject did during a task is useful, a test can further be utilized to determine what he or she was thinking while doing tasks of this sort. One route to insights about a subject's thoughts is to track a gaze of the subject while the subject is solving the maze. How far ahead of the pen are they looking? Does their gaze indicate when they realize they have turned down a path that does not lead to the exit? Does their gaze show us whether they plan ahead of choice points? What can gaze tell us about any other kinds of challenges the maze presents?

An embodiment of the invention includes maze solving using a writing implement that captures position in real time with eye tracking that captures gaze in real time. The combination of position and gaze data can be used to determine a cognitive status.

### 7.2.1 Example Embodiment

An example embodiment of the present invention is a system and method for calibrating cognitive load and detecting cognitive status. The example embodiment includes four components that interact synergistically:
a) a designed maze test;
b) an input device for writing that simultaneously tracks a position of a writing instrument with spatial and temporal accuracy, e.g., a stylus and electronic tablet, a digitizing ballpoint pen, or other such device;
c) a tracking device for tracking the subject's gaze with spatial and temporal resolution, enabling the determination of where on the test form they are looking; and
d) a processing module for analyzing pen position and eye position data, enabling such measurements as
   a. where was the subject's gaze relative to progress through the maze,
   b. in a situation where the subject retraces their path even though there have been no choices to make, what was the subject looking at that prompted him or her to do so,
   c. etc.

This invention builds on the ideas disclosed in U.S. Pat. No. 9,895,9085.

### 7.2.2 Example Test Form

Each test can include a calibration maze, a simple short straight channel through which the subject is asked to draw a line quickly. This serves both to accustom the user to the pen/stylus and provides a baseline measurement of their drawing speed in the absence of cognitive load.

Each test has two sub-tests. The first (the no-choice test) is a maze that, unknown to the subject, has no choice points, i.e., it is solved by simply following along through the only available path. The second maze (the choice test) is a variation on the first, constructed so that its solution is the same, but there are choices along the way. The subject is asked to do these in sequence, seeing only one of them at a time, and has no idea that the solution is the same for both of the test mazes.

The test can also include a number of mazes (for example, 3 mazes) intended to present different levels difficulty. The more advanced mazes have more choice points and may have embedded choices, i.e., a set of paths that all lead to dead ends but require multiple choices along the way to get there.

### 7.2.3 Example Testing Procedure

An example testing procedure includes subject performance of a calibration maze, then a no-choice maze, which is then removed from sight, and lastly, a choice maze. The calibration maze can provide measurement of the subject's movement speed. This differs even among normal individuals and may be substantially different for impaired persons. Given this measurement as a baseline of their movement speed, we can then distinguish e.g., what part of the subject's speed is due to cognitive load (having to find the solution path) vs due to simple muscle speed. In effect we are using each subject as their own control.

### 7.2.4 The Data Can Reveal Cognitive Status

The test form, data from a digital stylus and eye tracking, and analysis software together enable a variety of indicators of cognitive status and offer a novel view of maze use in cognitive testing.

For example, a difficulty of a maze, i.e., the cognitive load it presents, can be determined by more than just the total length of the path or the number of choices to be made. Subjects have been encountered who, when working their way through the no-choice maze, stop and begin to retrace their path, sometimes all the way back to the beginning of the maze, despite the fact that there have been no choices that could have been done differently. This has led to the observation that difficulty may also be determined by the character of the paths in between choice points. As a consequence, some mazes are designed to present varying kinds of paths, including some with relatively short straight segments, while others have considerably longer straight segments. This is a novel characterization of maze difficulty.

The ability to track both pen position and eye gaze position also provides a novel means of determining the level of difficulty the subject experiences, which may be different from a test designer's perceived difficulty. When, for example, the subject pauses drawing while working on the no-choice maze, little additional information from the pen may be obtained, but the subject's gaze can indicate what options he or she is exploring. For example: Is he or she looking ahead to see what's coming next, or looking further back to see whether they missed a choice, or other? As a consequence, it can be determined that the subject is experiencing a higher cognitive load at a point, and an indication of the nature of the difficulty can be obtained.

Other examples of this phenomenon include when a pen speed slows down for subjects with subtle cognitive impairment during a decision-making period, and it is possible to measure a location of decision-making by detecting those changes in pen speed. It is also possible to measure a level of perceived decision-making difficulty by magnitude of pen speed slow down, even if there are no errors in the maze. More impaired subjects may perceive more decision-making difficulty than healthy subjects, even when challenged with what may have originally been characterized as an “easy” decision.

### 7.3 Extracting Information about Cognitive State from a Symbol-Digit and/or Maze Test

Systems and methods including the Symbol-Digit tasks and Maze tests described above can measure features of human performance that are indicative of cognitive status, in particular healthy vs cognitively impaired statuses.

The method and system can include sensors, such as a digital pen and/or an eye tracking device, sampled a fixed frequency. For example, a digital pen can be included that measures its position 75 times a second. While these positions are a plausible approximation of the actual motion of the pen, they are at times too coarse, as for example where the pen path turns sharply. A cubic spline can adaptively be fit to the data, producing a smoother and more realistic motion path.

The systems and methods can include measurement of any combination of the following:
whether responses were centered in the answer space
pre-cell delay (the time between the end of the response in the previous cell and the start of the response in the next cell)
pre- and post-stroke rests, i.e., portions of a response where the pen is left basically immobile
the presence of inter-cell “hooklets”, i.e., sharp turns in a pen stroke occurring within a single response cell
the presence of cross-cell hooklets, i.e., those occurring from one response cell to the next
hooklets are classified as definite and possible
hooklet features including
   its length
   relative size
   pen speed
   “accuracy” of the hooklet as measured by
distance from the hooklet corner to the start of the next stroke
how close the projection of the hooklet comes to the start of the next stroke
   number of hooklets in each row, each task (i.e., translation vs copy) and each diagnostic class (e.g., healthy vs memory impairment).

Detected behaviors can include any combination of the following:
the presence of stray marks outside the answer cells
the presence of “thinking points”, i.e., very small strokes that appear to arise from the subject resting the pen on the paper while thinking about what to do next
differences in responses on the delayed recall part of the test when the test was given twice in succession to the same subject.

All these features can be used to derive indications of cognitive health, and their relative importance in contributing diagnostic information can be measured. In addition, these features permit measurements of cognitive load and may possibility reveal real-time learning, i.e., the increasing familiarity of the symbol-digit mapping over the course of the test itself.

In an number of embodiments described above, a machine-learning approach is used in which a number of joint stylus (or other drawing or pointing) input and gaze (or other eye tracking) input are processed to classify the subject according to one of a set of predefined categories and/or to make an assessment (e.g., output a numerical score) that matches a training corpus. In some embodiments described above, the input is segmented, and a “per-cell” feature vector may be used as a processed form of the joint input. In other embodiments, raw time-samples of the joint input may be used. In embodiments in which the test may vary from run to run (e.g., from subject to subject or between different runs with the same subject), a third input may correspond to the visual input to the subject. For example, a three-input embodiment may include a local maze structure near the stylus or the gaze location, the stylus location, and the gaze location. The machine learning approaches may use various techniques including neural networks (e.g., parameterized by trainable network weights), non-parametric statistical approaches (e.g., metric or nearest neighbor techniques characterized by training samples/exemplars), or parametric statistical approaches (e.g., parametric probabilistic models).

### 8. Implementations

The approaches described above can be implemented, for example, using a programmable computing system executing suitable software instructions or it can be implemented in suitable hardware such as a field-programmable gate array (FPGA) or in some hybrid form. For example, in a programmed approach the software may include procedures in one or more computer programs that execute on one or more programmed or programmable computing system (which may be of various architectures such as distributed, client/server, or grid) each including at least one processor, at least one data storage system (including volatile and/or non-volatile memory and/or storage elements), at least one user interface (for receiving input using at least one input device or port, and for providing output using at least one output device or port). The software may include one or more modules of a larger program. The modules of the program can be implemented as data structures or other organized data conforming to a data model stored in a data repository.

The software may be stored in non-transitory form, such as being embodied in a volatile or non-volatile storage medium, or any other non-transitory medium, using a physical property of the medium (e.g., surface pits and lands, magnetic domains, or electrical charge) for a period of time (e.g., the time between refresh periods of a dynamic memory device such as a dynamic RAM). In preparation for loading the instructions, the software may be provided on a tangible, non-transitory medium, such as a CD-ROM or other computer-readable medium (e.g., readable by a general or special purpose computing system or device), or may be delivered (e.g., encoded in a propagated signal) over a communication medium of a network to a tangible, non-transitory medium of a computing system where it is executed. Some or all of the processing may be performed on a special purpose computer, or using special-purpose hardware, such as coprocessors or field-programmable gate arrays (FPGAs) or dedicated, application-specific integrated circuits (ASICs). The processing may be implemented in a distributed manner in which different parts of the computation specified by the software are performed by different computing elements. Each such computer program is preferably stored on or downloaded to a computer-readable storage medium (e.g., solid state memory or media, or magnetic or optical media) of a storage device accessible by a general or special purpose programmable computer, for configuring and operating the computer when the storage device medium is read by the computer to perform the processing described herein. The system may also be considered to be implemented as a tangible, non-transitory medium, configured with a computer program, where the medium so configured causes a computer to operate in a specific and predefined manner to perform one or more of the processing steps described herein.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

What is claimed is:

1. A method of determining a cognitive assessment for a subject comprising:
receiving input position data associated with input provided by the subject during a time that the subject is responding to a cognitive test;
receiving gaze position data associated with a gaze of the subject during the time that the subject is responding to the cognitive test; and
determining a cognitive assessment for the subject based at least in part on the input position data and the gaze position data.

2. The method of claim 1 wherein the input position data and the gaze position data are aligned to a common timeline.

3. The method of claim 1 wherein the input position data includes a time series of input positions and the gaze position data includes a time series of gaze positions.

4. The method of claim 1 wherein determining the cognitive assessment includes processing the input position data and the gaze position data using a parameterized transformation.

5. The method of claim 4 further comprising pre-processing the input position data and the gaze position data according to data characterizing the cognitive test prior to using the parameterized transformation.

6. The method of claim 4 wherein the parameterized transformation includes a neural network.

7. The method of claim 1, wherein the cognitive test is a symbol-digit test.

8. The method of claim 7, wherein determining the cognitive feature includes measuring a period of time for which the subject gazed at a stimulus symbol in the symbol-digit test, detecting whether the subject gazed at a key of the symbol-digit test, detecting whether the subject gazed at a prior stimulus item, detecting whether the subject gazed at a position or a feature of the displayed test, measuring a period of time for which the subject gazed at a key of the symbol-digit test, measuring a period of time for which the subject obtained a correct pairing or an incorrect pairing, or any combination thereof.

9. The method of claim 7, wherein the symbol-digit test includes a symbol-digit decoding task.

10. The method of claim 7, wherein the symbol-digit test includes a digit-digit copying task.

11. The method of claim 1, wherein the cognitive test is a maze test.

12. The method of claim 11, wherein determining the cognitive feature includes measuring a position of the subject's gaze, comparing the position of the subject's gaze to a position of an input provided by the subject, determining whether the subject pauses, determining whether the subject retraces a path, or any combination thereof.

13. The method of claim 11, wherein the maze test is a no-choice test.

14. The method of claim 11, wherein the maze test includes a no-choice subtest.

15. The method of claim 11, wherein the maze test is a choice test.

16. The method of claim 11, wherein the maze test includes a choice subtest.

17. The method claim 1, wherein the cognitive test is displayed on a surface and the writing instrument is a stylus to which the surface is responsive.

18. The method of claim 17, wherein the surface is a tablet computer interface, a wall, or a virtual surface.

19. The method of claim 1, wherein the cognitive test is displayed on a physical or electronic page and the writing instrument is a digitizing pen.

20. The method of claim 1, wherein the cognitive test includes subtests of varying cognitive loads.

21. The method of claim 1, further comprising changing a visual appearance of a stimulus of the cognitive test.

22. The method of claim 21, wherein changing the visual appearance of the stimulus includes producing a change in cognitive load or perceived cognitive load.

23. The method claim 21, further comprising determining an impact of the changed cognitive load based on a detected gaze.

24. The method of claim 1, further comprising displaying the cognitive test to a subject.

25. The method of claim 1 wherein determining the cognitive assessment for the subject based at least in part on the input position data and the gaze position data includes determining at least part of the cognitive assessment while the subject is still responding to the cognitive test.

26. A system for determining a cognitive assessment for a subject comprising:
an input for receiving input position data associated with input provided by the subject during a time that the subject is responding to a cognitive test;
an input for receiving gaze position data associated with a gaze of the subject during the time that the subject is responding to the cognitive test; and
one or more processors for determining a cognitive assessment for the subject based at least in part on the input position data and the gaze position data.

27. A non-transitory computer-readable medium having encoded thereon a sequence of instructions which, when loaded and executed by a processor, causes the processor to perform a method for determining a cognitive assessment for a subject by:
receiving input position data associated with input provided by the subject during a time that the subject is responding to a cognitive test;
receiving gaze position data associated with a gaze of the subject during the time that the subject is responding to the cognitive test; and
determining a cognitive assessment for the subject based at least in part on the input position data and the gaze position data.

28. A method for determining parameters for a parameterized transformation to be used in a cognitive health assessment system, the method comprising:
receiving input position data associated with input provided by a plurality of subjects during times that the subjects are responding to a cognitive test;
receiving gaze position data associated with a gaze of the plurality of subjects during the times that the subjects are responding to the cognitive test;
receiving cognitive health assessment label data associated with cognitive health assessments determined from a performance of the plurality of subjects on the cognitive test; and
estimating parameters for the parameterized transformation based at least in part on the input position data, the gaze position data, and the cognitive health assessment label data, wherein the parameterized transformation is configured to accept input position data for a subject responding to the cognitive test, gaze position data for the subject responding to the cognitive test, and produce a cognitive health assessment for the subject.
